(19) United States
(12) Patent Application Publication
(10) Pub. No.: US 2025/0134370 A1
(43) Pub. Date: May 1, 2025

(54) MEASURING DEVICE AND AN ILLUMINATION DEVICE

(71) Applicant: CANON VIRGINIA, INC., Newport News, VA (US)

(72) Inventors: Takayuki Sugiyama, Utsunomiya (JP); Norihiko Utsunomiya, Newport News, VA (US); Makoto Fukuhara, Yorktown, VA (US)

(21) Appl. No.: 18/933,235

(22) Filed: Oct. 31, 2024

Related U.S. Application Data
(60) Provisional application No. 63/594,755, filed on Oct. 31, 2023.

(51) Int. Cl. A61B 3/12; A61B 3/14

(52) U.S. Cl. CPC A61B 3/12; A61B 3/14

(57) ABSTRACT

Provided are a measuring device and an illumination device that may include a light source, one or more optical elements configured to polarize light from the light source, at least one imaging element, and an optical system configured to form an image of a retina illuminated by light polarized by the one or more optical elements, wherein the image is formed in the at least one imaging element. Light polarized by the one or more optical elements may include at least a first illumination light with a first polarization state and a second illumination light with a second polarization state, and at least a part of an illumination region of the first illumination light and an illumination region of the second illumination light are different.

### CROSS-REFERENCE TO RELATED APPLICATIONS

The present application claims benefit of and priority to U.S. provisional application No. 63/594,755 filed Oct. 31, 2023, the disclosure of which is hereby incorporated by reference in its entirety for all purposes. Priority benefit is claimed under 35 U.S.C. § 119(e).

### BACKGROUND INFORMATION

### Field of Disclosure

The present disclosure relates to an illumination device and a measuring device equipped with the same.

### Description of Related Art

The fovea (depression) exists in the center of the macula of the eye's retina and it is known that the best visual acuity is achieved in the fovea. It is also known that by detecting the birefringence state caused by the Henle fibers surrounding the fovea, it is possible to determine where the subject is staring (fixation state).

WO 99/20173 and U.S. Pat. No. 10,111,584 provide an ophthalmic nerve scanner (measuring device) having a projection device (illumination device) that projects a projection image (illumination image) onto the retina of the eye and a photodetector that acquires a reflection image showing the fixation state of the eye reflected by the retina. The photodetector disclosed in WO 99/20173 separates the polarization information from the light reflected by the retina by a polarization beam splitter (PBS) and acquires the respective intensities. In addition, the photodetector disclosed in Patent Document U.S. Pat. No. 10,111,584 is arranged in a position conjugate to the retina of the eye and detects the light reflected from the retina of the eye as a two-dimensional image.

However, in the measuring device disclosed in the aforementioned Patent WO 99/20173, as shown in FIG. 9, the optical path splits in a 90° direction when separating the polarization by a PBS, so it is necessary to arrange a plurality of sensors for each polarization.

A similar configuration is required when using this configuration in U.S. Pat. No. 10,111,584 and, in addition, it will take the shape of additionally arranging a plurality of sensors for obtaining information from the left and right eyeballs.

That is, in such a configuration, the arrangement becomes complicated, and it is necessary to suppress their relative misalignment and the like in order to prevent deterioration of the detection accuracy, and high position accuracy is required. As a result, the measuring device becomes more complex and larger.

Therefore, to overcome the shortcomings of conventional systems, the following embodiments provide reduced size measuring devices with a simple configuration.

### SUMMARY OF THE DISCLOSURE

An aspect of the present disclosure provides a measuring devices that includes a light source, one or more optical elements that are configured to polarize light from the light source, a pair of imaging elements, and an optical system configured to form an image of a retina illuminated by light polarized by the one or more optical elements, wherein the image is formed in a respective imaging element of the pair of imaging elements. Light polarized by the one or more optical elements includes at least a first illumination light with a first polarization state and a second illumination light with a second polarization state, and at least a part of an illumination region of the first illumination light and an illumination region of the second illumination light are different.

Another aspect of the present disclosure provides a measuring device that includes a light source, one or more optical elements configured to polarize light from the light source, at least one imaging element, and an optical system in which the optical element forms an image of the retina illuminated by light polarized by the one or more optical elements. Light polarized by the one or more optical elements includes at least a first illumination light with a first polarization state and a second illumination light with a second polarization state, and at least a part of the illumination region of the first illumination light and the illumination region of the second illumination light are different.

A further aspect of the present disclosure provides an illumination device for measuring a retina utilizing an imaging device, with the illumination device including a light source and one or more optical elements configured to polarize light from the light source, with the light polarized by the one or more optical elements includes at least a first illumination light with a first polarization state and a second illumination light with a second polarization state, and with at least a part of an illumination region of the first illumination light and an illumination region of the second illumination light being different.

According to one of more of the above configurations, it is possible to provide a small measuring device with a simple configuration.

### BRIEF DESCRIPTION OF THE DRAWINGS

The accompanying drawings, which are incorporated herein and form part of the specification, illustrate various embodiments, objects, features, and advantages of the present disclosure.

FIG. 1 is a schematic diagram of the main part showing a basic configuration of a measuring device equipped with an illumination device according to an embodiment.

FIG. 2 is a schematic diagram of the main part of the illumination device according to Embodiment 1.

FIG. 3 is a configuration diagram of a light source unit according to Embodiment 1.

FIG. 4 is a diagram showing the optical components' arrangement of the illumination device according to Embodiment 1.

FIG. 5 is a schematic diagram of the main part of the illumination device according to Embodiment 2.

FIG. 6 is a diagram showing the optical components' arrangement of the illumination device according to Embodiment 2.

FIG. 7 is a schematic diagram of the main part of the illumination device according to Embodiment 3.

FIG. 8 is a diagram showing the optical components' arrangement of the illumination device according to Embodiment 3.

FIG. 9 is a diagram showing the optical components' arrangement of the illumination device according to Embodiment 4.

FIG. 10 is a schematic diagram of the main part of the illumination device according to Embodiment 5.

FIG. 11 is a schematic diagram of the main part of the imaging device according to the present Embodiment.

FIG. 12 is a schematic diagram of a conventional measuring device.

FIG. 13A-B is a schematic diagram of an imaging optical system according to the present disclosure.

Throughout the figures, the same reference numerals and characters, unless otherwise stated, are used to denote like features, elements, components or portions of the illustrated embodiments, and duplicate descriptions are omitted. Each drawing may, for convenience sake, be drawn on a scale different from the actual scale. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

### Embodiment 1

### Measurement Device

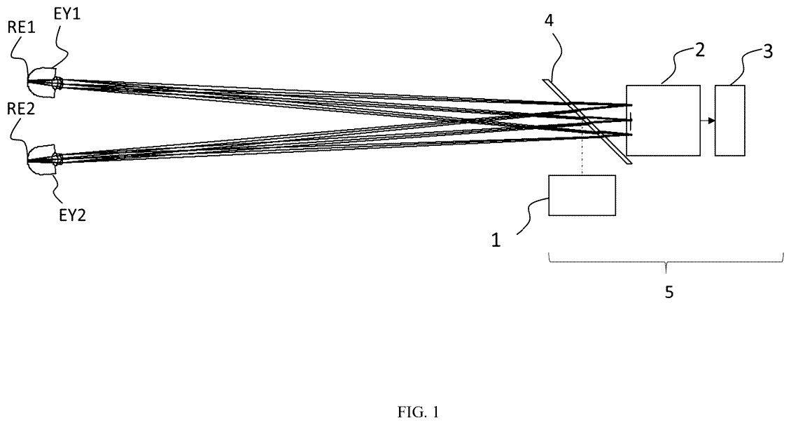

FIG. 1 is a schematic diagram of the main part showing a basic

configuration of a measuring device 5 (a fixation measuring device) equipped with an illumination device according to an embodiment of the present disclosure.

The measuring device 5 comprises an illumination device 1, an imaging device 2, and an arithmetic part 3.

The illumination device 1 is equipped with a light source part and a plurality of optical elements to form an illumination image. Reflect the light of the formed illumination image by a half mirror 4 and guide it toward the retina RE1 and RE2 of the subject's eyes EY1 and EY2, respectively. At this time, the illumination image formed by the illumination device has a conjugate relationship with the retina RE1 and RE2, and illuminates the left and right eyes simultaneously. In this example, set the wavelength of the light source part of the illumination device from 800 to 900 nm, considering the absorption of light by water and the specific visual sensitivity of the human eye. Because the light measures the retina via the cornea, vitreous, and lens of the eye, water, which is the main component of these tissues, absorbs and attenuates it. The transmissivity of light relative to water is wavelength dependent and has a high transmissivity in the wavelength range of approximately 200 nm to 900 nm. On the other hand, the human eye has a sensitivity characteristic for each wavelength of light called specific visual sensitivity, which senses light from approximately 360 nm to 800 nm. For more stable measurements, one can avoid the above sensitivity range to prevent miosis. Based on the above point of view, also considering the absorption of water, this practical example describes an example that uses light in the 800 nm to 900 nm range as a light source for the illumination device. However, this example does not exclude wavelengths outside the 800 to 900 nm range, and for example, the wavelength of the light source unit of the illumination device may be 780 to 930 nm.

Reflected light from the retina RE1 and RE2, which are conjugate to the illumination image, is transmitted through the half mirror 4 and guided to the imaging device 2. The imaging device 2 is equipped with an imaging optical system and at least one imaging element, and images the guided reflected light. In addition, the imaging device 2 images information related to the polarization of incident light to the retina and reflected light from the retina. Moreover, the imaging device 2 simultaneously photographs reflected light from the left and right eyes of the subject.

The arithmetic part 3 is equipped with a means for measuring the fixation state of the subject based on information reflected from the retina RE1 and RE2 of both eyes.

### Illumination Device

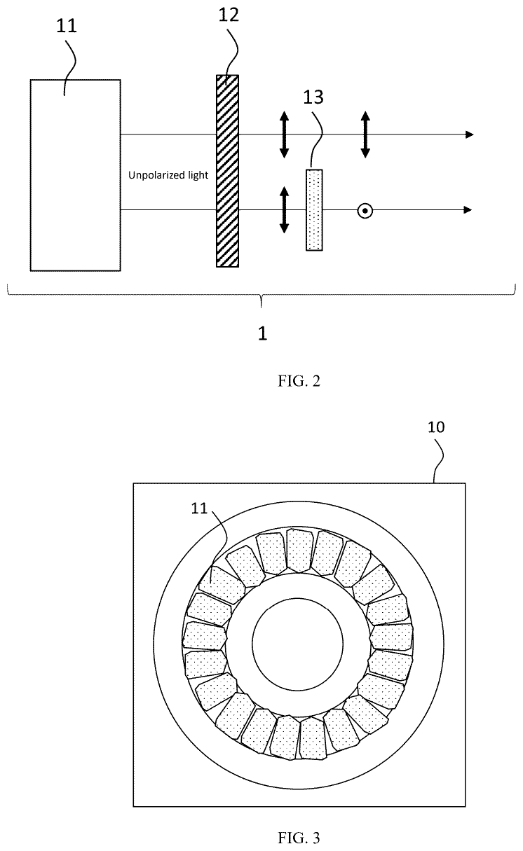

The following describes the illumination device according to this practical example in more detail. FIG. 2 is a schematic diagram of the main part of the illumination device 1 according to Embodiment 1. The illumination device 1 is composed of at least one light source part 11, an optical element 12, and an optical element 13.

The light source part 11 of Embodiment 1 consists of LEDs, which irradiate light in the direction of the arrows shown in FIG. 2. Light emitted from the light source part 11 passes through the optical element 12. Here, the optical element 12 has a polarizer arranged to adjust the polarization state of light in a certain direction. Since the polarization state of the light emitted from the LEDs of the light source part 11 is in a non-polarized state, it is possible to produce light in a certain unidirectional linear polarization state by passing it through the polarizer of the optical element 12. The optical element 12 can be composed of any type of polarizer, such as a crystal type such as quartz, a glass type such as an optical multilayer film, or a resin sheet type made by orienting compound molecules. Next, pass a part of the light that has passed through the polarizer of the optical element 12 through the optical element 13. Here, for the optical element 13, a ½ wavelength plate that can produce a linear polarization state rotated by 90° from a linear polarization state in a certain direction is arranged. As a result, the portion that does not pass through the ½ wavelength plate of the optical element 13 is light in a linear polarization state in one direction, and the portion that has passed through is light in a linear polarization state rotated 90°. This enables converting light emitted from one set of LEDs into light with two different polarization states.

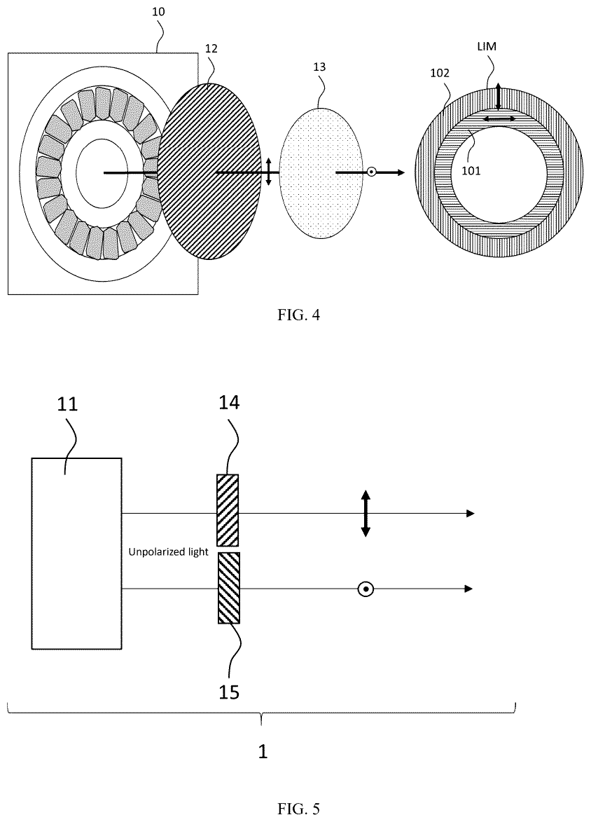

The following describes the illumination image formed by the illumination device 1 using FIG. 3 and FIG. 4. FIG. 3 is a configuration diagram of the light source unit 10. Arrange the LEDs, which make up the light source part 11 described above, into a ring shape. Arranging it in the shape of a ring makes it possible to form an illumination image with a ring-shaped illumination region.

In the case of Embodiment 1, as shown in FIG. 4, make the radii of the optical element 12 polarizer and the optical element 13 ½ wavelength plate different for the light source part 11, and arrange the centers in agreement. Hereby, it is possible to form an illumination image LIM having ring-shaped illumination regions 101 and 102 with two types of polarization states with coincident centers and different radii. By illuminating the retina RE1 and RE2, which are in a conjugate relationship with this ring-shaped illumination image, it is possible to image information on different polarization states at once.

### Imaging Device

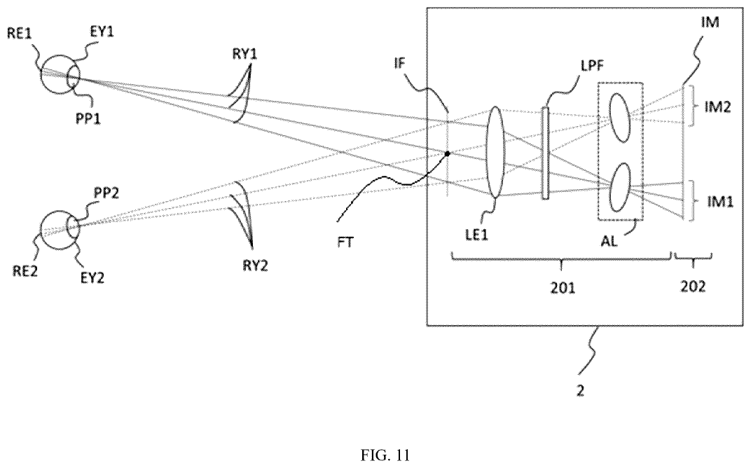

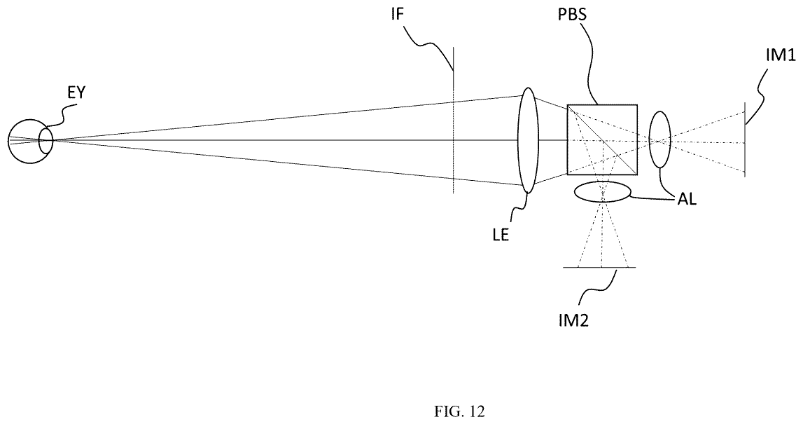

This embodiment commonly adopts the following imaging optical system. FIG. 11 shows a schematic diagram of the main part of the imaging device 2.

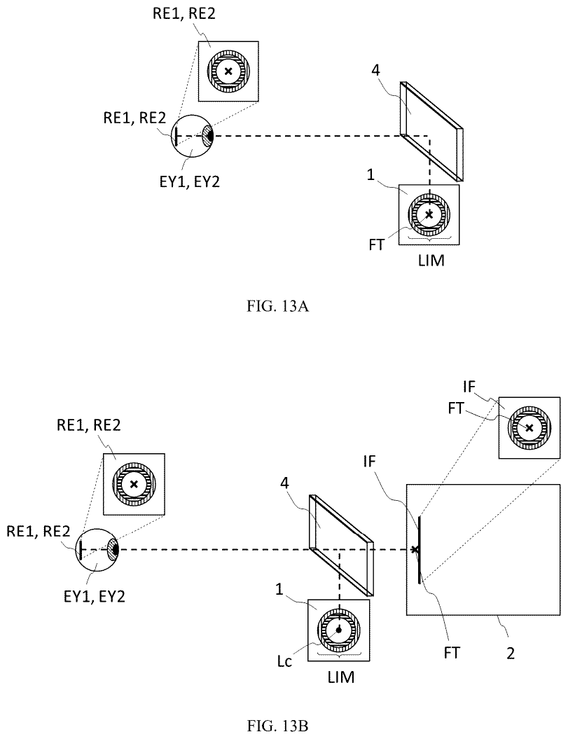

The imaging device 2 consists of an imaging optical system 201 and at least one imaging element 202, as described above. The imaging optical system 201 is composed of an optical system LE1, a long-pass filter LPF, and an array optical system AL. The imaging surface IM corresponds to the light receiving surface of an imaging device 2 such as a CCD (Charge Coupled Device) sensor or a CMOS (Complementary Metal Oxide Semiconductor) sensor. The retina RE1 and RE2 of the eyes EY1 and EY2 respectively are in a conjugate relationship with the intermediate image IF, and the intermediate image IF is in turn in a conjugate relationship with the imaging plane IM via the imaging optical system 201. In order to make the illumination image LIM and the retina RE1 and RE2 into a conjugate relationship, use a fixation lamp FT. Since the image produced by the fixation lamp FT is formed with visible light, the subject can verify it. Arrange the fixation lamp FT on the same plane as the illumination image LIM, as shown in FIG. 13a, or on the same plane as the intermediate image IF, as shown in FIG. 13b. It is possible to make the illumination image LIM consisting of infrared light and the subject's retina into a conjugate relationship by the subject staring at the image of the fixation lamp FT. In addition, it is possible to fix the visual axis to the center of the illumination image LIM by arranging the fixation lamp FT at a position corresponding to the center Lc of the illumination image LIM in the relevant plane. The fixation lamp FT may be a marker made of LEDs that is a self-luminescent element, an organic EL element or a liquid crystal display element with back illumination, or a fluorescent dye excited by another excitation light source, etc.

RY1 represents light reflected from the retina RE1, and RY2 represents light reflected from the retina RE2. The reflected lights RY1 and RY2 form intermediate image IF, which is then captured by the imaging element 202 on respective imaging surface IM1/IM2. Here, IM1 and IM2 can be imaged by a single CCD or CMOS sensor, or can be imaged separately by a pair of CCD or CMOS sensors. Each optical system of the array optical system AL is placed in an approximately conjugate position with respect to the pupil (lens) PP1 of the eye EY1 and the pupil PP2 of the eye EY2, respectively. Here, both the optical system LE1 and the array optical system AL have positive power. In addition, by placing the long-pass filter LPF on the optical path, it is possible to suppress stray light caused by external light or the like.

Due to the formation of an illumination image in which light in different polarization states turns into different illumination regions in the illumination device 1 of the present disclosure, there is no need to arrange an optical system to separate the polarized light in the imaging device 2. Therefore, the imaging device can be considerably smaller than the conventional example, and it is possible to miniaturize the measuring device.

### EMBODIMENT 2

FIG. 5 is a schematic diagram of the main part of the illumination device of Embodiment 2. As for the measuring device, the description is omitted, because it is similar to Embodiment 1. In Embodiment 2, unlike Embodiment 1, the configuration is such that the light emitted from the LEDs of the light source part 11 enters the different polarizers 14 and 15, respectively. The polarizer 14 is a polarizer similar to the one of Embodiment 1, so arrange it in a position that allows some of the light from the LEDs to pass through. In addition, some of the remaining light also passes through the polarizer 15 arranged adjacent to the polarizer 14. The polarizer 15 is the polarizer 14 rotated in a 90° direction. It can generate light in a linear polarization state with a different 90° direction with respect to the light in a linear polarization state in one direction that has passed through the polarizer 14, and can form an illumination image with two different types of polarization states same as Embodiment 1. The polarizer 14 and the polarizer 15 can be any type, such as a crystal type such as quartz, a glass type such as optical multilayer film, or a resin sheet type made by orienting compound molecules.

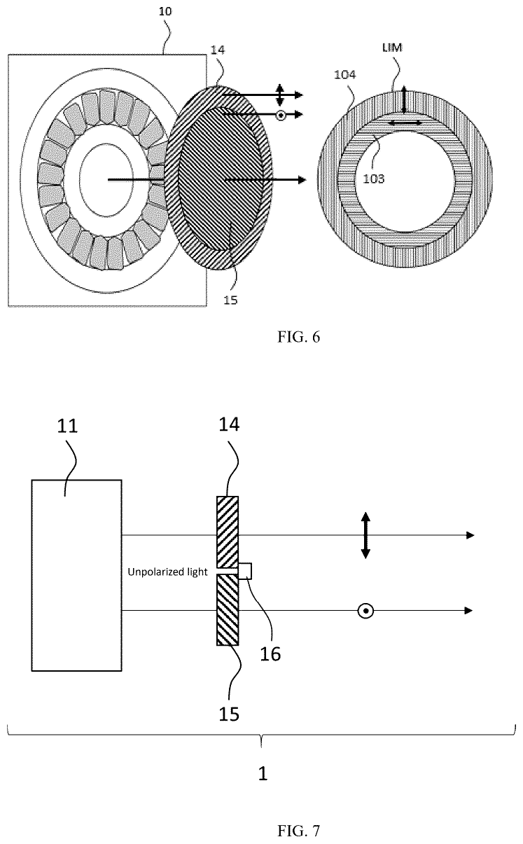

In case of the present embodiment, arrange the polarizer 14 and the polarizer 15 in concentric circles as shown in FIG. 6. Here, the polarizer 14 has an opening in the center so that light passing through the polarizer 15 does not pass through the polarizer 14. This makes it possible to form an illumination image LIM having ring-shaped illumination regions 103 and 104 with two types of polarization states with coincident centers and different radii. By illuminating the retina RE1 and RE2, which are in a conjugate relationship with this ring-shaped illumination image, it is possible to image information of different polarization states at once.

### Embodiment 3

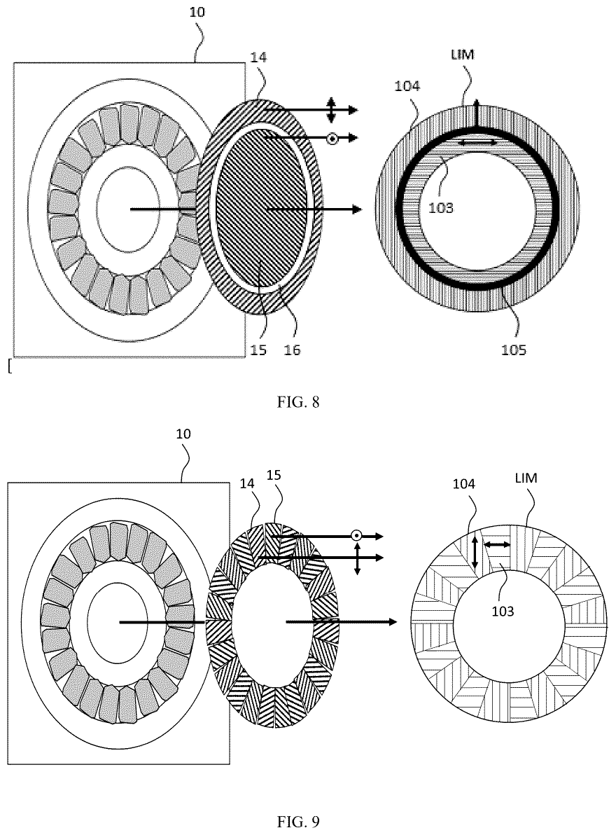

FIG. 7 is a schematic diagram of the main part of the illumination device of Embodiment 3. As for the measuring device, the description is omitted because it is similar to Embodiment 1 and 2. This embodiment is almost the same configuration as Embodiment 2, but it places a light shielding part 16 at the boundary between the polarizer 14 and the polarizer 15. The light shielding part 16 can be composed of any type of light shielding material, such as light-shielding cloth, processed paper, board, aluminum foil, masking tape or an optical member. By providing the light shielding part 16, it becomes possible to improve the separation accuracy of the polarized light. It becomes possible by reducing the crosstalk near the boundary between the light passing through the polarizer 14 in a certain unidirectional linear polarization state and the light passing through the polarizer 15 in a different 90° directional linear polarization state with respect to the light. FIG. 8 shows an arrangement example of the optical components in this embodiment. Arrange the light shielding part 16 to cover the boundary between the polarizer 14 and the polarizer 15, that is, the inner edge of the polarizer 14 and the outer edge of the polarizer 15. Alternatively, the light shielding part 16 may be formed by arranging the polarizer 14 and the polarizer 15 so they overlap each other. This shields the boundaries of the ring-shaped illumination regions 103 and 104, which have two types of polarization states with different radii, and along with reducing the crosstalk of each polarization signal; it clearly divides the illumination regions, to enable facilitating detection. As in Embodiment 1, by illuminating the retina RE1 and RE2, which are in a conjugate relationship with the illumination image LIM formed by the illumination regions 103 and 104, information of different polarization states can be imaged at once.

### Embodiment 4

FIG. 9 shows a schematic diagram of the main part of the illumination device of Embodiment 4. In this embodiment, the polarizers 14 and 15 are arranged alternately close to each other in the circumferential direction in which the LEDs are arranged in ring shapes. As a result, it is possible to form an illumination image LIM having illumination regions 103 and 104 whose centers coincide and have two different types of polarization states within the ring-shaped illumination region, and which are alternately close in the circumferential direction of the ring. As in Embodiment 1, by illuminating the retina RE1 and RE2, which are in a conjugate relationship with the ring-shaped illumination image, it is possible to image information of different polarization states all at once. cl Embodiment 5

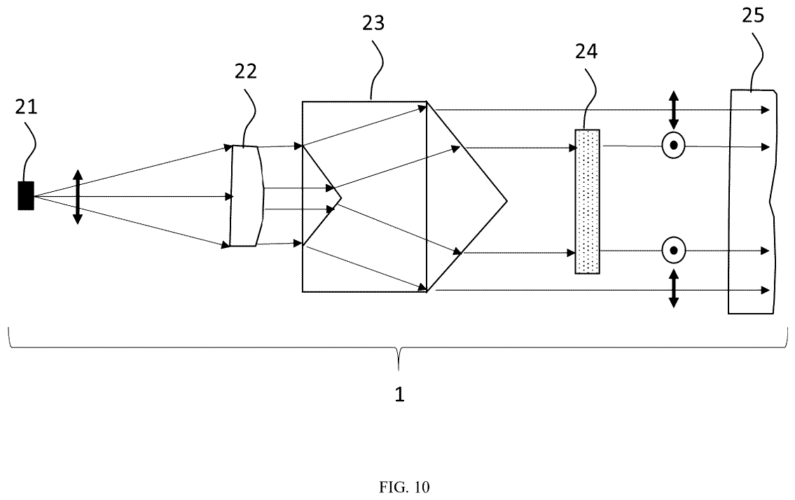

FIG. 10 is a schematic diagram of the main part of the illumination device of Embodiment 5.

The light source part 21 of this Embodiment 5 is a laser light source and emits light in the direction of the arrows shown in FIG. 10. The light emitted from the laser light source 21 becomes parallel light due to the lens 22. The parallel light is incident on the Axicon lens 23 and forms a ring-shaped light. Because the laser light source 21 is in a linearly polarized state, the light that has passed through the Axicon lens 23 becomes a ring-shaped linearly polarized light. Next, a part of the light that has passed through the Axicon lens 23 passes through the optical element 24. Here, for the optical element 24, a ½ wavelength plate that can produce a linear polarization state rotated 90° from a certain unidirectional linear polarization state, is arranged. This is the same method as in Embodiment 1. Thus, the portion not passing through the ½ wavelength plate of the optical element 24 becomes light in a certain unidirectional linear polarization state, and the portion that passes through becomes light in a linear polarization state rotated 90°. The ½ wavelength plate 24 has a radius smaller than the radius of the Axicon lens 23 to allow the ring-shaped inner light coming through the Axicon lens 23 to pass through. After that, pass it through the toroidal lens 25 to form an illumination image. Thus, it is possible to turn the light emitted from one laser light source 21 into a ring shape and form an illumination image of the light in two different types of polarization states with different illumination regions. Here too, as in Embodiment 1, by illuminating the retina RE1 and RE2, which are in a conjugate relationship with this ring-shaped illumination image, it is possible to image information of different polarization states at once.

As explained above, the measuring device or the illumination device described in the embodiments 1 to 5 comprises one or more optical elements that polarize light from the light source. The optical element includes at least one of one or more polarizers and/or a ½ wavelength plate.

For example, the optical element includes a polarizer and a ½ wavelength plate, and a part of the luminous flux that has passed through the polarizer passes through the ½ wavelength plate. Alternatively, the optical elements include 2 polarizers whose polarization directions differ from each other, and a part of the luminous flux from the LEDs passes through one polarizer and a different luminous flux from the partial luminous flux passes through the other polarizer.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. Skilled artisans may employ such variations as appropriate, and the present disclosure may be practiced other than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### Reference Number Listing

1 Illumination Device
2 Imaging Device
3 Arithmetic Part
4 Half Mirror
5 Measuring Device
10 Light Source Unit
11, 21 Light Source Part
12, 13, 24 Optical Element
101, 102, 103, 104 Illumination Region
22 Lens
23 Axicon Lens
25 Toroidal Lens
201 Imaging Optical System
202 Imaging Element

What is claimed is:

1. A measuring device comprising:
a light source;
one or more optical elements configured to polarize light from the light source;
a pair of imaging elements; and
an optical system configured to form an image of a retina illuminated by light polarized by the one or more optical elements, wherein the image is formed in a respective imaging element of the pair of imaging elements,
wherein light polarized by the one or more optical elements includes at least a first illumination light with a first polarization state and a second illumination light with a second polarization state, and
wherein at least a part of an illumination region of the first illumination light and an illumination region of the second illumination light are different.

2. A measuring device comprising:
a light source;
one or more optical elements configured to polarize light from the light source;
at least one imaging element; and
an optical system configured to form an image of a retina illuminated by light polarized by the one or more optical elements, wherein the image is formed in the at least one imaging element,
wherein light polarized by the one or more optical elements includes at least a first illumination light with a first polarization state and a second illumination light with a second polarization state, and
wherein at least a part of an illumination region of the first illumination light and an illumination region of the second illumination light are different.

3. The measuring device according to claim 2,
wherein the illumination region of the first illumination light and the illumination region of the second illumination light have a ring shape,
wherein centers of the illumination region of the first illumination light and the illumination region of the second illumination light coincide, and
wherein radii of the illumination region of the first illumination light and the illumination region of the second illumination light are different.

4. The measuring device according to claim 2, wherein the illumination region of the first illumination light and the illumination region of the second illumination region are alternately formed in a circumferential direction of the ring shape.

5. The measuring device according to claim 2, further comprising a light shielding part positioned at a boundary between the illumination region of the first illumination light and the illumination region of the second illumination light.

6. The measuring device according to claim 2, wherein the first illumination light and the second illumination light are linearly polarized illumination lights with 90° differing directions.

7. The measuring device according to claim 2, wherein the measuring device is configured to simultaneously illuminate retinas of each of a left eyeball of a user and a right eyeball of the user.

8. The measuring device according to claim 2,
wherein the illumination region of the first illumination light and the illumination region of the second illumination light have a ring shape,
wherein the light source includes a fixation lamp, and
wherein a center of the ring shape and a center of an image of the fixation lamp coincide.

9. The measuring device according to claim 2, further comprising:
a fixation lamp arranged on a plane of an intermediate image conjugate to the illumination region of the first illumination light, the illumination region of the second illumination light, the retina, and an imaging plane of the at least one imaging element,
wherein the illumination region of the first illumination light and the illumination region of the second illumination light have ring shapes whose centers coincide with each other, and
wherein a position of a ring shape in the intermediate image coincides with a center of the fixation lamp.

10. The measuring device according to claim 2,
wherein the light source comprises a plurality of LEDs,
wherein the one or more optical elements include a polarizer and a ½ wavelength plate, and
wherein at least a part of luminous flux that has passed through the polarizer passes through the ½ wavelength plate.

11. The measuring device according to claim 2,
wherein the light source comprises a plurality of LEDs,
wherein the one or more optical elements include at least two polarizers having polarization directions that differ from each other, and
wherein at least a part of luminous flux from the plurality of LEDs passes through one polarizer of the at least two polarizers and at least a part of different luminous flux from the partial luminous flux passes through an other polarizer of the at least two polarizers.

12. The measuring device according to claim 2, wherein the light source is formed as a plurality of LED light sources arranged in a ring shape.

13. The measuring device according to claim 2,
wherein the light source is a laser,
wherein the one or more optical elements include an Axicon lens and a ½ wavelength plate, and
wherein at least a part of luminous flux that has passed through the Axicon lens passes through the ½ wavelength plate.

14. The measuring device according to claim 2, wherein light output by the light source has a wavelength of 800 to 900 nm.

15. The measuring device according to claim 2, wherein a conjugate intermediate image is formed on an imaging surface of the retina and the at least one imaging element.

16. The measuring device according to claim 2, wherein a light emitting surface of the light source is positioned conjugate to the retina.

17. An illumination device for measuring a retina utilizing an imaging device, the illumination device comprising:
a light source; and
one or more optical elements configured to polarize light from the light source,
wherein the light polarized by the one or more optical elements includes at least a first illumination light with a first polarization state and a second illumination light with a second polarization state, and
wherein at least a part of an illumination region of the first illumination light and an illumination region of the second illumination light are different.

18. The illumination device according to claim 17,
wherein the one or more optical element include at least one of one or more a polarizers and/or a ½ wavelength plate.

19. The illumination device according to claim 18,
wherein the light source comprises a plurality of LEDs,
wherein the one or more optical elements include a polarizer and a ½ wavelength plate, and
wherein at least a part of luminous flux that has passed through the polarizer passes through the ½ wavelength plate.

20. The illumination device according to claim 18,
wherein the light source comprises a plurality of LEDs,
wherein the one or more optical elements include two polarizers having polarization directions that differ from each other, and
wherein at least a part of luminous flux from the plurality of LEDs passes through one polarizer of the at least two polarizers and at least a part of different luminous flux from the partial luminous flux passes through an other polarizer of the at least two polarizers.
